(19) United States
(12) Patent Application Publication
(10) Pub. No.: US 2024/0191892 A1
(43) Pub. Date: Jun. 13, 2024

(54) AIR PURIFIER

(71) Applicant: DEFOND ELECTECH CO., LTD, Dongguan (CN)

(72) Inventors: TAK CHUEN OR, Dongguan (CN); CHENG CHEN NIEH, Dongguan (CN); CHIU KEUNG LOONG, Dongguan (CN); KIN YU WONG, Dongguan (CN); CHUN KAN LIN, Dongguan (CN); LAP YI LAW, Dongguan (CN)

(73) Assignee: DEFOND ELECTECH CO., LTD, Dongguan (CN)

(21) Appl. No.: 18/164,698

(22) Filed: Feb. 6, 2023

(30) Foreign Application Priority Data
Dec. 12, 2022 (CN) 202223319157.X

(51) Int. Cl. F24F 8/15; F24F 8/22

(52) U.S. Cl. CPC F24F 8/15; F24F 8/22

(57) ABSTRACT

The present application provides an air purifier comprising a main body, wherein the main body comprises: a housing, wherein an interior of the housing is hollow, an air inlet is formed at one end of the housing, an air outlet is formed at the other end of the housing, and positions of the air inlet and the air outlet are interchangeable; a fan mounted in the housing and used for sucking outside air from one end of the housing or for sucking outside air from the other end of the housing; a photocatalyst member mounted in the housing; and ultraviolet light-emitting members mounted in the housing, so that ultraviolet light emitted by the ultraviolet light-emitting members can effectively irradiate the photocatalyst member.

### CROSS-REFERENCE TO RELATED APPLICATIONS

The application claims priority to Chinese patent application No. 202223319157.X, filed on Dec. 12, 2022, the entire contents of which are incorporated herein by reference.

### TECHNICAL FIELD

The present application relates to the technical field of air purification, and in particular to an air purifier.

### BACKGROUND

With the rapid development of economy, the continuous progress of society and the continuous improvement of living standard, the attention of people to the air quality is continuously enhanced. In real life, people work in all walks of life and harvest the fruits of labor, but at the same time, they also cause serious pollution to air. Most of pollutants mixed in the air are particles, dust and part of toxic and harmful gases, which accompany each corner of living space of people and seriously affect the health of people. At the same time, natural dust, such as pollen, produced by non-human labor in life also causes problems to human health. In order to reduce the impact of gas pollution on human beings, it is necessary to purify the air in a living environment of people.

As automobiles are one of vehicles commonly used by people, the quality of air inside the automobile has a great impact on the health of people. The air purification of the automobile includes two parts, namely, the purification of the air outside the automobile, and the purification of circulating air inside the automobile. Pollution sources of the air outside the automobile are mainly dust, exhaust gas emitted by the automobiles on roads, etc. Pollution sources of the air inside the automobile mainly are engine exhaust gas entering the automobile through gaps at the bottom of the automobile, sweat and carbon dioxide produced by human bodies, a smoke smell released by smoking, etc. The polluted air can cause adverse effects on the health of people and also can lead people to be tired, thus easily causing traffic accidents and greatly reducing the comfort performance of automobiles.

An existing vehicle-mounted air purification device uses a single purification member, is poor in purification effect, and fails to achieve a good effect of purifying the air. Moreover, it can only purify the air inside the automobile, but cannot purify the air outside the automobile.

Chinese utility model patent publication No. CN205871686U discloses a vehicle-mounted air purification device. The vehicle-mounted air purification device has a hollow housing, an air filtering member and an adsorption member, wherein the housing is mounted on an outer surface of the top of a vehicle, an air inlet is formed at a front end of the housing, and an air outlet is formed at a rear end of the housing, the air filtering member and the adsorption member are sequentially provided inside the housing along a flow direction of the air in the housing to sequentially treat the air. The vehicle-mounted air purification device of this patent is mounted on the outer surface of the top of the vehicle, positions of the air inlet and the air outlet are fixed, and the vehicle-mounted air purification device can only be mounted in a specified direction, so that the use is not convenient.

### SUMMARY

In order to overcome the shortcomings and deficiencies existing in the prior art, the purpose of the present application is to provide an air purifier.

The purpose of the present application is realized by the following technical solution: an air purifier comprises a main body, wherein the main body comprises:
a housing, wherein an interior of the housing is hollow, end plates are fixed at two ends of the housing, a plurality of airflow holes are formed in the end plates, an air inlet is formed at one end of the housing, an air outlet is formed at the other end of the housing, and positions of the air inlet and the air outlet are interchangeable;
a fan mounted in the housing and used for sucking outside air from one end of the housing or for sucking outside air from the other end of the housing;
a photocatalyst member mounted in the housing;
ultraviolet light-emitting members mounted in the housing, so that ultraviolet light emitted by the ultraviolet light-emitting members effectively irradiate the photocatalyst member.

Preferably, the photocatalyst member is in a sheet-like structure, which is transversely mounted in a middle of the housing, the ultraviolet light-emitting members are in two groups, and the two groups of ultraviolet light-emitting members are respectively mounted above and below the photocatalyst member.

Preferably, the air purifier further comprises functional members, wherein the functional members are mounted at one end of the main body and/or the other end of the main body, and a plurality of vent holes are formed in two ends of the functional members.

Preferably, the functional members comprise at least one of a fragrance member, a filtering member and an adsorption member.

Preferably, cross-sectional shapes of the functional members are the same as that of the main body.

Preferably, a plurality of buffer columns are provided at outer side surfaces of the end plates and the functional members in an outward protruding manner, a plurality of buffer grooves matched with the buffer columns are provided at inner side surfaces of the functional members, and the buffer columns are embedded in the buffer grooves.

Preferably, clamping pieces are provided at the inner side surfaces of the functional members in an inward protruding manner, and clamping grooves matched with the clamping pieces are formed in the outer side surfaces of the end plates and the functional members, and the clamping pieces are clamped into the clamping grooves.

Preferably, the main body further comprises a control panel which is electrically connected with the fan and the ultraviolet light-emitting members.

Preferably, a charging interface and a charging indicator lamp are provided on a side surface of the housing, and the charging interface and the charging indicator lamp are electrically connected with the control panel respectively.

Preferably, the photocatalyst member is a transparent porous photocatalyst member that is in a hood-shaped structure, a plurality of ventilation holes are formed in two ends of the hood-shaped structure, and the ultraviolet light-emitting members are mounted in the hood-shaped structure.

The present application has the beneficial effects that: the air purifier of the present application adopts the fan to suck the outside air and provide an air flow, and degrades, via the photocatalyst member, toxic and harmful pollutants such as formaldehyde and ammonia mixed in the air passing through its surface to achieve the effect of disinfecting the air, meanwhile, the air purifier can effectively kill various bacteria, decompose and harmlessly treat toxins released by the bacteria or fungi, so that the purification effect of the air purifier is enhanced; the light emitted by the ultraviolet light-emitting members can be utilized to sterilize the air and provide a light source for the photocatalyst member, thus improving the pollutant degradation efficiency of the photocatalyst member and further strengthening the purification effect of the air purifier of the automobile.

The main body of the air purifier of the present application is symmetrical, the housing with the hollow interior is adopted, the air inlet is formed at one end of the housing, the air outlet is formed at the other end of the housing, positions of the air inlet and the air outlet are interchangeable and the air purifier can be mounted in two directions, so that the use is convenient.

### BRIEF DESCRIPTION OF DRAWINGS

FIG. 1 is a perspective view of embodiment 1 of the present application.

FIG. 2 is an exploded perspective view of embodiment 1 of the present application.

FIG. 3 is a sectional view of aside surface of embodiment 1 of the present application.

FIG. 4 is a sectional view of functional members described in the present application when assembled.

FIG. 5 is a structural diagram of a side surface of the present application.

FIG. 6 is a structural diagram of a bottom surface of the present application.

FIG. 7 is an exploded perspective view of embodiment 2 of the present application.

### NUMERAL REFERENCE

main body 1, housing 11, air inlet III, air outlet 112, charging interface 113, charging indicator lamp 114, mounting hole 115, foot pad 116, end plate 12, airflow hole 121, fan 13, photocatalyst member 14, ventilation hole 141, ultraviolet light-emitting member 15, control panel 16, functional member 2, vent hole 21, buffer column 22, buffer groove 23, clamping piece 24, U-shaped elastic sheet 241, clamping block 242, pressing sheet 243, pressing block 244, inclined surface 245, clamping groove 25, stop block 251, notch 252.

### DETAILED DESCRIPTION OF THE EMBODIMENTS

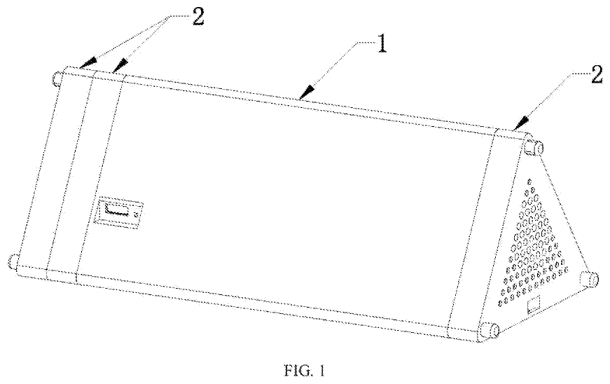

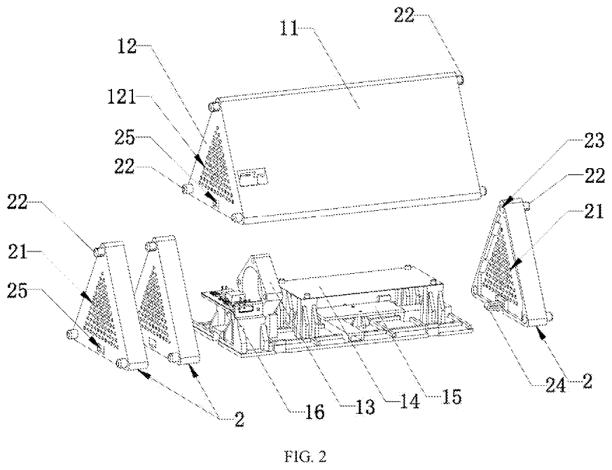

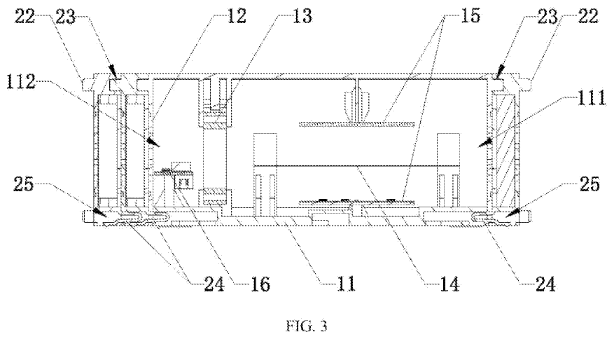

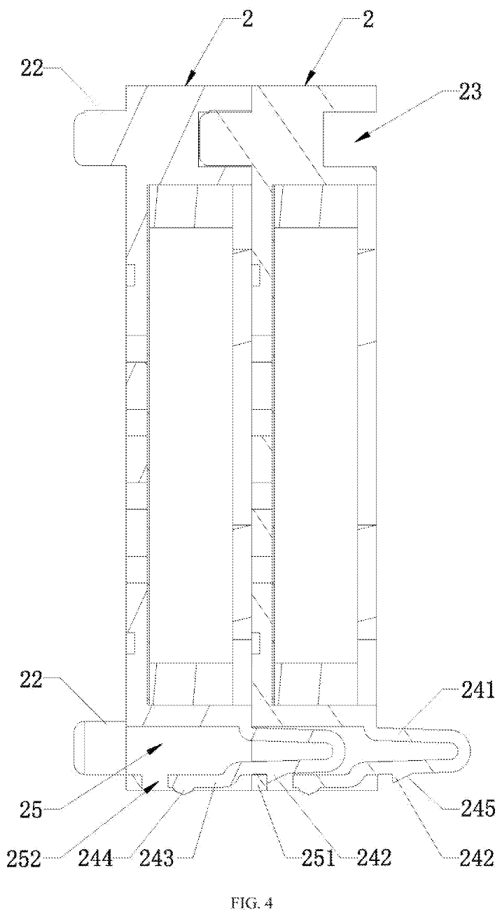

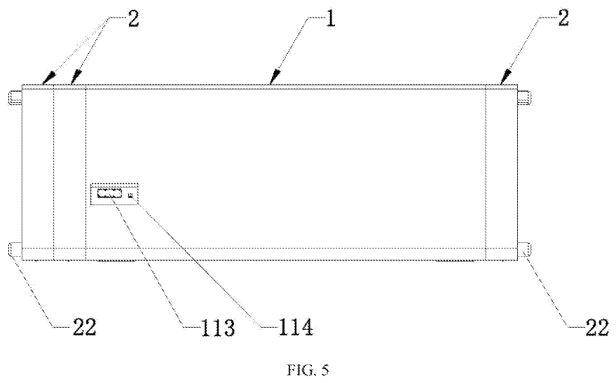

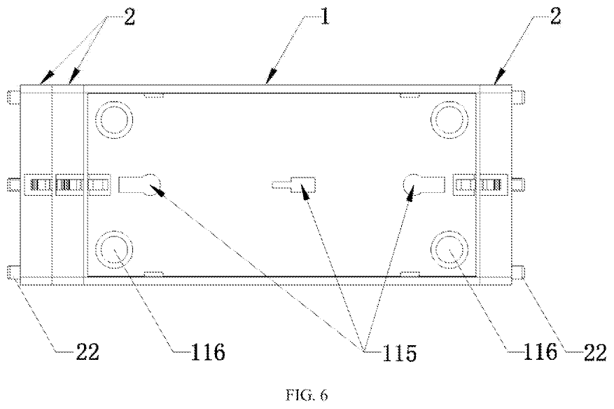

In order to facilitate the understanding of those skilled in the art, the present application will be further explained with reference to embodiments and FIGS. 1-7, and reference to the embodiments is not intended to limit the present application.

### Embodiment 1

Referring to FIGS. 1-6, an embodiment of the present application provides an air purifier comprising a main body 1, wherein the main body 1 comprises:
a housing 11, wherein end plates 12 are fixed at two ends of the housing 11, a plurality of airflow holes 121 are formed in the end plates 12, an air inlet 111 is formed at one end of the housing 11, an air outlet 112 is formed at the other end of the housing 11, and positions of the air inlet 111 and the air outlet 112 are interchangeable;
a fan 13 mounted in the housing 11 and used for sucking outside air from one end of the housing 11 or for sucking outside air from the other end of the housing 11;
a photocatalyst member 14 mounted in the housing 11;
ultraviolet light-emitting members 15 mounted in the housing 11, so that ultraviolet light emitted by the ultraviolet light-emitting members 15 can effectively irradiate the photocatalyst member 14.

In addition, in the above-mentioned embodiment, the photocatalyst member 14 is in a sheet-like structure, which is transversely mounted in a middle of the housing 11, the ultraviolet light-emitting members 15 are in two groups, and the two groups of ultraviolet light-emitting members 15 are respectively mounted above and below the photocatalyst member 14. The sheet-like photocatalyst member 14 can be photocatalyst cloth, a photocatalyst film, a photocatalyst filter screen, etc., the two groups of ultraviolet light-emitting members 15 are mounted above and below the sheet-like photocatalyst member 14, which can fully irradiate an upper surface and a lower surface of the sheet-like photocatalyst member 14, thereby improving the pollutant degradation efficiency of the photocatalyst member 14 and further strengthening a purification effect of the air purifier of a vehicle.

In addition, in the above-mentioned embodiment, the air purifier further comprises functional members 2, wherein the functional members 2 are mounted at one end of the main body 1 and/or the other end of the main body 1, and a plurality of vent holes 21 are formed in two ends of the functional members 2. The type and number of the functional members 2 can be added as needed, and one or more functional members can be added, and can be mounted at one end of the main body 1 and/or the other end of the main body 1.

In addition, in the above-mentioned embodiment, the functional members 2 comprise at least one of a fragrance member, a filtering member and an adsorption member. The fragrance member is used to improve the smell of air in the vehicle, thereby enhancing the comfort of an inner space of the vehicle; the filtering member can filter and remove particulate pollutants suspended in the air, and can effectively filter and purify fine particles such as smoke and dust, the filtering member such as a filter screen and filter cotton; and the adsorption member can selectively adsorb toxic and harmful gas components contained in the air and perform certain purification treatment on the air, the adsorption member such as activated carbon and a molecular sieve.

Specifically, in this embodiment, three functional members 2 are used, wherein one of the functional members being a filtering member is mounted at one end of the main body 1, and the other two of the functional members being fragrance members are mounted at the other end of the main body 1, so as to improve the purification effect of the air purifier and the smell in the vehicle. It is easy to understand that the filtering member and the adsorption member are mounted at the air inlet 111 of the main body 1, and the fragrance member is mounted at the air outlet 112 of the main body 1; when the main body 1 is reversed, that is, the positions of the air inlet 111 and the air outlet 112 are interchanged, at this time, the fan 13 needs to rotate reversely, so that the fan 13 sucks outside air from the other end of the housing 11.

In addition, in the above-mentioned embodiment, cross-sectional shapes of the functional members 2 are the same as that of the main body 1. In this embodiment, the cross sections of the functional members 2 and the cross section of the main body 1 are triangular, and the shapes of the functional members 2 are consistent with that of the main body 1, so that the mounting and use of the functional members 2 are facilitated. The shapes of the cross sections of the functional members 2 and the main body 1 can also be round, oval or other regular polygons, and any obvious substitutions are within the scope of the present application without departing from the concept thereof.

In addition, in the above-mentioned embodiment, a plurality of buffer columns 22 are provided at outer side surfaces of the end plates 12 and the functional members 2 in an outward protruding manner, a plurality of buffer grooves 23 matched with the buffer columns 22 are formed in inner side surfaces of the functional members 2, and the buffer columns 22 are embedded in the buffer grooves 23. The arrangement of the buffer columns 22 on the outer side can prevent the air inlet 111 and the air outlet 112 from being blocked.

In addition, in the above-mentioned embodiment, clamping pieces 24 are provided at inner side surfaces of the functional members 2 in an inward protruding manner, clamping grooves 25 matched with the clamping pieces 24 are formed in outer side surfaces of the end plates 12 and the functional members 2, and the clamping pieces 24 are clamped into the clamping grooves 25. The arrangement of the clamping between the functional members 2 and the end plates 12 and the clamping between the functional members 2 can ensure stable connection between the functional members 2 and the main body 1, also facilitate disassembly, and facilitate replacement of the functional members 2 after the purification effect decreases due to long working time of the functional members 2.

Specifically, each clamping piece 24 comprises a U-shaped elastic sheet 241, a clamping block 242 fixed at a bottom of the U-shaped elastic sheet 241, a pressing piece 243 fixed at the end of the bottom of the U-shaped elastic sheet 241, and a pressing block 244 fixed at the bottom of the pressing piece 243; a stop block 251 is fixed in the clamping groove 25 at a position corresponding to the clamping block 242, an inclined surface 245 is provided at one side of the clamping block 242, the other side of the clamping block 242 is clamped with the stop block 251, and a notch 252 is formed in the bottom of the clamping groove 25 at a position corresponding to the pressing block 244. Since one side of the clamping block 242 is the inclined surface 245, during mounting, the stop block 251 presses the inclined surface 245 of the pressing block 244, the U-shaped elastic sheet 241 is in a pressed state, and after the pressing block 244 passes through the stop block 251, the U-shaped elastic sheet 241 recovers its deformation, so that the other side of the clamping block 242 is clumped with the stop block 251; during dismounting, it is only necessary to press the pressing block 244, and the U-shaped elastic sheet 241 is in a pressed state, so that the clamping block 242 can exit from the stop block 251. The arrangement of the above structure facilitates the mounting and dismounting of the functional members 2.

In addition, in the above-mentioned embodiment, the main body 1 further comprises a control panel 16, the control panel 16 being electrically connected with the fan 13 and the ultraviolet light-emitting members 15. The control panel 16 is used to control the fan 13 and the ultraviolet light-emitting members 15, and provide power to the fan 13 and the ultraviolet light-emitting members 15, the ultraviolet light-emitting members 15 being ultraviolet lamps or containing a plurality of ultraviolet light-emitting diodes.

In addition, in the above-mentioned embodiment, a charging interface 113 and a charging indicator lamp 114 are provided on a side surface of the housing 11, and the charging interface 113 and the charging indicator lamp 114 are electrically connected with the control panel 16, respectively. The charging interface 113 is a type-C charging interface or other charging interface 113 for charging, and the charging indicator lamp 114 is used to indicate a charging status.

In addition, in the above-mentioned embodiment, a plurality of mounting holes 115 are formed in a bottom surface of the housing 11, and the shapes of the mounting holes 115 are different. The arrangement of the mounting holes 115 facilitates mounting the air purifier on a vehicle window. A plurality of foot pads 116 are fixed at the bottom surface of the housing 11, and the foot pads 116 are provided to reduce the vibration of the main body 1 and prolong the service life.

### Embodiment 2

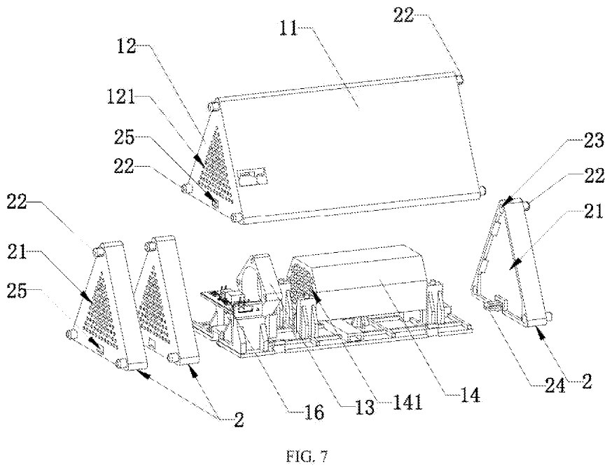

As shown in FIG. 7, an embodiment of the present application provides another air purifier, which differs from that of embodiment 1 in that the photocatalyst member 14 is a transparent porous photocatalyst member in a hood-shaped structure, a plurality of ventilation holes 141 are formed in two ends of the hood-shaped structure, and the ultraviolet light-emitting members 15 are mounted in the hood-shaped structure. The ultraviolet light-emitting members 15 are mounted in the hood-shaped structure, and the ultraviolet light-emitting members 15 irradiate the transparent porous photocatalyst member of the hood-shaped structure, thereby improving the pollutant degradation efficiency of the photocatalyst member 14 and further strengthening the purification effect of the air purifier of the vehicle.

The above-mentioned embodiments are preferred implementations of the present application. Besides, the present application can be implemented in other ways, and any obvious substitutions are within the protection scope of the present application without departing from the concept thereof.

What is claimed is:

1. An air purifier comprising a main body, wherein the main body comprises:
a housing, wherein an interior of the housing is hollow, end plates are fixed at two ends of the housing, a plurality of airflow holes are formed in the end plates, an air inlet is formed at one end of the housing, an air outlet is formed at the other end of the housing, and positions of the air inlet and the air outlet are interchangeable;
a fan mounted in the housing and used for sucking outside air from one end of the housing or for sucking outside air from the other end of the housing;
a photocatalyst member mounted in the housing;
ultraviolet light-emitting members mounted in the housing, so that ultraviolet light emitted by the ultraviolet light-emitting members effectively irradiate the photocatalyst member.

2. The air purifier according to claim 1, wherein the photocatalyst member is in a sheet-like structure, which is transversely mounted in a middle of the housing, the ultraviolet light-emitting members are in two groups, and the two groups of ultraviolet light-emitting members are respectively mounted above and below the photocatalyst member.

3. The air purifier according to claim 1, wherein the air purifier further comprises functional members, wherein the functional members are mounted at one end of the main body and/or the other end of the main body, and a plurality of vent holes are formed in two ends of the functional members.

4. The air purifier according to claim 3, wherein the functional members comprise at least one of a fragrance member, a filtering member and an adsorption member.

5. The air purifier according to claim 3, wherein cross-sectional shapes of the functional members are the same as that of the main body.

6. The air purifier according to claim 5, wherein a plurality of buffer columns are provided at outer side surfaces of the end plates and the functional members in an outward protruding manner, a plurality of buffer grooves matched with the buffer columns are provided at inner side surfaces of the functional members, and the buffer columns are embedded in the buffer grooves.

7. The air purifier according to claim 5, wherein clamping pieces are provided at the inner side surfaces of the functional members in an inward protruding manner, and clamping grooves matched with the clamping pieces are formed in the outer side surfaces of the end plates and the functional members, and the clamping pieces are clamped into the clamping grooves.

8. The air purifier according to claim 1, wherein the main body further comprises a control panel which is electrically connected with the fan and the ultraviolet light-emitting members.

9. The air purifier according to claim 8, wherein a charging interface and a charging indicator lamp are provided on a side surface of the housing, and the charging interface and the charging indicator lamp are electrically connected with the control panel respectively.

10. The air purifier according to claim 1, wherein the photocatalyst member is a transparent porous photocatalyst member that is in a hood-shaped structure, a plurality of ventilation holes are formed in two ends of the hood-shaped structure, and the ultraviolet light-emitting members are mounted in the hood-shaped structure.
